(19) United States
(12) Patent Application Publication
(10) Pub. No.: US 2004/0224029 A1
(43) Pub. Date: Nov. 11, 2004

(54) Particle possessing a membrane

(76) Inventor: Wolfgang Geiger, Ansbach (DE)

Correspondence Address: HARNESS, DICKEY & PIERCE, P.L.C., P.O.BOX 8910, RESTON, VA, 20195, US

(21) Appl. No.: 10/815,670

(22) Filed: Apr. 2, 2004

(30) Foreign Application Priority Data
Apr. 4, 2003 (DE) 10315538.4

(51) Int. Cl. A61K 9/14

(52) U.S. Cl. 424/489

(57) ABSTRACT

A particle can be transported in a fluid stream, in particular a bloodstream. It includes a membrane which encloses a particle core. The membrane contains a number of functional elements, which are integrated in a matrix and which, in dependence on the concentration of a body substance, bring about substance transport through, and/or substance accumulation at, the membrane.

The present application hereby claims priority under 35 U.S.C. §119 on German patent application number DE 103 15 538.4 filed Apr. 4, 2003, the entire contents of which are hereby incorporated herein by reference.

1. Field of the Invention

The invention generally relates to a particle which can be transported in a fluid stream, in particular a human or animal bloodstream, preferably for analytical, diagnostic and/or therapeutic applications.

2. Background of the Invention

A particle is disclosed, for example, in DE 691 26 535 T2. Further, U.S. Pat. No. 4,329,332 A discloses particles which have a diameter of less than 1 μm, which are formed from a polymerized material and which contain a biologically active substance.

Information on these medically utilizable particles, which are also termed nanocapsules, is also contained in the dissertation entitled “NMR-Untersuchungen an Nanokapsel-Dispersionen [NMR analyses of nanocapsule dispersions]” (Dirk Hoffmann, Duisburg University, Chemistry, 4.9.2000). This document describes spherical hollow objects which are to be used in medicine as tissue-specific active compound-carrier systems as being nanocapsules.

An active compound which is enclosed in the nanocapsule is surrounded by polymeric scaffolding. The aim is to use these objects to create a drug-targeting system which encloses the active compound and transports it, while being protected in this manner, to the target tissue, where it is able to exert its curative effect without harming the remainder of the body.

In connection with an intravenous application, the diameter of the active compound-carrier systems should be less than 4 μm so as to ensure that they are able to migrate through the smallest blood vessels in the body and cannot give rise to any embolisms which would be life-threatening to patients. An active compound-carrier system can, for example, possess, in the form of a vesicle, a membrane lamella which is spherically closed on itself and which is composed of a lipid bilayer.

As artificially prepared vesicles, liposomes typically have a diameter of between 20 nm and 3 μm and a membrane having a thickness of approx. 5 nm. Pharmaceutical applications of liposomes are based, in particular, on the possibility of encapsulating hydrophilic molecules in their aqueous internal space. As a result of this encapsulation, the liposome serves as a permeation barrier having a delayed release effect.

As an alternative to liposomes, it is also possible to prepare, as active compound-carrier systems, solid lipid nanoparticles (SLN), particularly from physiological lipids or from lipids composed of physiological components. In order to achieve a targeting effect in the case of these solid lipid nanoparticles, the nanocapsule wall is surrounded by a stabilizing surfactant layer. However, when effected in this way, specific release of the active compound at the desired site is only possible under certain circumstances.

Biologically effective microcapsules which are prepared by enclosing biological cells in an envelope composed of biocompatible polymer materials are described, for example, in DE 102 03 628 A1. These microcapsules contain fused biological cells, with it being possible to carry out a fusion, i.e. an electrofusion, in an electrical field. Such a fusion can have the advantage that it is possible to control the number of cells which are to be fused. The fusion is preferably carried out prior to the encapsulation, i.e before the cells are enclosed in the polymer material.

Microparticles or nanoparticles, in particular for cosmetic or pharmaceutical compositions, are also disclosed in DE 199 32 216 A1. This document describes the possibility of using porous particles to remove molecules from a liquid medium, to “harvest” them, or to release enclosed molecules slowly at an active site. The pH dependence of the solubility of given compounds can, inter alia, be used to exert an influence on the long-term release or delayed release of an enclosed molecule.

DE 693 29 295 T2 describes polymer microspheres having a diameter of less than 180 μm which are used for the controlled release of growth hormones. According to this document, the long-term release or prolonged release of a drug, as influenced, inter alia, by the nature of one or more polymer compositions, takes place continuously or discontinuously and linearly or nonlinearly.

Biocompatible microcapsules which are described in DE 691 26 535 T2, and which are envisaged for transplantation into an animal, possess a membrane having more than one layer. In this case, the outermost, polycationic layer of the membrane is crosslinked ionically with another membrane layer and composed of a water-soluble nonionic polymer. The microcapsules are said to have a surface which is resistant to cell adhesion and to contain cells which are able to receive nutrients and signal molecules and produce a desired product.

DE 37 82 840 T2 discloses a drug in the form of a microcapsule which provides delayed release and whose envelope is, for example, formed, inter alia, from cellulose acetate phthalate. Enzymes are able to influence the disintegration of the microcapsule, which encloses a fine-grained core consisting of a water-soluble medicament.

### A SUMMARY OF THE INVENTION

n embodiment of the invention is based on an object of specifying a particle which can be transported in a fluid stream, in particular the bloodstream of a human being or an animal, and whose effect is in particular exerted selectively at the desired site, in particular in the desired tissue. It is furthermore an object of an embodiment of the invention to specify an analytical and/or diagnostic method which uses such a particle.

According to an embodiment of the invention, an object may be achieved by way of a particle and by way of a method. The particle which can be transported in a fluid stream, preferably a human or animal bloodstream, possesses a membrane which encloses a particle core and which has a number of functional elements which are integrated in a matrix and which, in dependence on the concentration of a body substance, bring about a substance transport through the membrane and/or an accumulation of substance on the membrane. In this connection, a substance transport through the membrane can take place inwards and/or outwards. Functional elements are, in particular, portal elements, for example ion channels, and/or detectors, in particular for detecting the body substance which is present in a medium surrounding the particle. The matrix has the task, in particular, of fixing the functional element to the particle core and/or of sealing off the particle core, where appropriate together with the functional element(s), from the exterior. The particle core can, for example, be filled with a drug and/or possess a cavity for receiving a substance from the medium surrounding the particle.

According to a preferred embodiment, the particle is intended to be used in a diagnostic method. In this connection, an endogenous substance is, in dependence on the concentration of a body substance in the medium surrounding the particle, accumulated on the membrane, incorporated into the membrane and/or transported through the membrane into the particle core. In general, this is based on an internalized receptor, channel or exchange membrane being functional.

With regard to the principle of the mode of functioning of receptors, the reader is referred, by way of example, to tyrosine kinase receptors. The endogenous substance which has accumulated in or on the particle is not necessarily identical to the body substance whose concentration has an influence on the accumulation or incorporation process. The particle is, in particular, suitable for selectively gathering a substance which is present at low concentration in a fluid, for example blood, serum, plasma, urine, sputum, cerebrospinal fluid or another animal or human body fluid.

However, the particle can likewise also be used for selectively gathering up one or more substances from solutions, liquids, liquid wastes, extracts or other liquid analytical samples, for example beverages. The substance which is present in the medium, which is not necessarily fluid, surrounding the particle and whose concentration has an influence on the properties of the membrane, is uniformly designated as the body substance, irrespective of the nature of the medium.

The selective gathering-up of the human or other substance replaces an enrichment or concentration step which is otherwise necessary. Whereas, for example when a blood analysis was being carried out, it would only be possible, in accordance with the prior art, to quantitatively determine a highly dilute substance using a blood sample of relatively high volume, the enrichment of the substance to be analysed on the particles which were used for this purpose in a diagnostic method would at least make it possible to reduce the sample volume substantially.

When the particle is used to accumulate an endogenous substance, the latter is not necessarily incorporated in the particle, or accumulated on the particle, in unaltered form. In every case, however, the particle is altered in a detectable manner. The detection of the particle, which is, for example, transported in a bloodstream, is preferably effected without any physical sampling, i.e., for example, withdrawal of blood. For this purpose, at least the shape which the particle assumes after having accumulated the endogenous substance can be displayed using an imaging method.

Medicoinstrumental methods which can suitably be used in this connection are, in particular, ultrasonic methods, NMR methods, CT (computer tomography) investigations, fluorescence measuring techniques and proton emission tomography. By coupling the use of the particle to these imaging medicoinstrumental methods, it is possible to implement what is overall an electronic diagnostic concept. The IT component is also termed in-vivo logistics intelligence (transportomics). As is explained in more detail below, this can also be integrated into a therapeutic concept which makes use of the properties of the particle which can be influenced from the exterior, i.e. extracorporeally.

The particle, which, depending on its dimensions, is also termed a nanoparticle, can also be envisaged, in addition or as an alternative to the accumulation of an endogenous substance, for releasing a substance, in particular a drug. In this connection, the rate at which the drug is released depends on the concentration of a body substance in the medium surrounding the particle. The release of the drug can, for example, be effected by the drug being conveyed within the matrix by a functional element which functions as a portal element or by the membrane being completely disintegrated, with the particle core, which contains the drug or which is identical to the drug, being released simultaneously. When the membrane disintegrates in dependence on the concentration or the presence of the body substance, the membrane then acts as a whole as a functional element for releasing the drug.

The drug can not only be present in the particle core but can, instead of or in addition to this, also be present in the membrane. In this connection, the membrane can, as in all the other implementation examples as well, be composed of a single layer or of several layers. In the case of a multilayer membrane, in which a drug is integrated, the latter is preferably located in the inner layer or in the inner layers of the membrane. When the particle is used diagnostically or therapeutically, it is, in particular, its biocompatibility and biodegradability which are relevant.

The biological half-life of the particle should be sufficiently long to achieve an adequate accumulation effect when the particle is being used diagnostically and, when it is being used therapeutically, to avoid an administration of the medicament which is too frequent and may possibly be stressful to patients. In order to adequately take both aspects into account, preference is given to the membrane being designed to be subject to attack by enzymes in the human or animal body over the medium to long term, i.e. to the particle being broken down within a period of at least a few hours, preferably a few days or weeks. In general, the particle core can be broken down, for example by means of being metabolized, more rapidly than the membrane.

According to a preferred further development, the particle core includes or forms a reaction region which is envisaged for transforming the substance. This applies both in connection with taking up the substance and in connection with releasing the substance into or out of the particle core. In the latter case, the particle core does not contain the drug in the form in which it is to be used but, instead, only at least one precursor.

The conversion of this precursor into the drug is triggered by a signal which acts on the particle. This signal can be provided by the concentration of a body substance in the medium surrounding the particle. In general, a biochemical signal or biochemical reaction releases a precursor from the particle and local enzymes then convert this precursor into the desired product. An enzymic reaction consequently brings about a prodrug-drug conversion.

The activity of the particle can be influenced by an extracorporeal signal. Such an extracorporeal signal can be provided in the form of a physical force or of a field, for example an ultrasonic irradiation, or of a magnetic field. When the particle is used dermatologically, it is also possible to use light, preferably light in a given limited wavelength range, for example infrared light, to effect an activation, i.e. selective conversion, substance release and/or substance uptake. For this purpose, the membrane possesses a receptor which reacts to the appropriate electromagnetic radiation and which influences the permeability of the membrane and/or a substance conversion in the particle core. The receptor can be integrated into a matrix as a functional element within the membrane or itself form the entire membrane.

The fact that the activity of the particle can be controlled from the exterior affords the possibility of, instead of introducing a drug into the body, only introducing the information which specifies which substance is produced in the body, at what time it is produced and where it is produced. As long as this information, which is fed into the body by way, for example, of an ultrasonic signal or an IR signal or by way of a magnetic field, is not imparted, the particle can be constituted in such a way that it behaves passively, i.e. in a biologically neutral manner, or only displays a limited basal activity. What is termed an IT pathway logistics module can consequently be used to control a therapy from the exterior and the therapy can also be coupled to diagnostic methods, as a result of which it is possible, taken overall, to implement a closed loop. The drug which is to be released by the particle can be administered in an individualized manner using what is termed an in-vivo transport system, i.e. both the therapeutic use and the diagnostic use can be personalized.

A particularly advantageous use of the particle can be achieved when its reaction region, which forms a part of the particle core or is identical to this core, is designed for transforming an endogenous, i.e. human or animal, intermediate. This use comes into consideration when the human or animal organism is itself no longer able to produce a necessary end substance from the intermediate. Provided the rate at which the intermediate is produced in the organism depends on the need for the end substance which is ultimately required, the transformation of the intermediate to the end substance in the particle results in the production of the end substance being self-regulating. Coupling the reaction to the endogenous intermediate matches both the quantity of the end substance which is discharged from the particle and the positional distribution of the end substance to the actual need.

The dimensions of the particle are selected within a wide range, in accordance with the given requirements. The external diameter is preferably between 50 nm and 10 μm, in particular between 200 nm and 2 μm, while the thickness of the membrane is between 2 nm and 1 μm, in particular at most 100 nm. A polymer, preferably a biologically degradable polymer, is preferably selected as the material for the membrane, in particular for the matrix which embeds the functional elements. This polymer may incorporate electronic components in the form of a polymer electronic system, in particular for a bidirectional data link with external IT components. Such a polymer electronic membrane preferably forms the interface with imaging medicoinstrumental methods.

### BRIEF DESCRIPTION OF THE DRAWINGS

The present invention will become more fully understood from the detailed description of preferred embodiments given hereinbelow and the accompanying drawings, which are given by way of illustration only and thus are not limitative of the present invention.

In that which follows, one exemplary embodiment of the invention is explained in move detail with the aid of the drawings. In these drawings, and in each case in greatly simplified diagrammatic representation:

FIG. 1 shows a particle together with an internalized endogenous substance,

FIG. 2 shows a particle together with an endogenous substance which is bound to a membrane,

FIG. 3 shows a particle together with a drug which can be released from this particle,

FIG. 4 shows a particle together with a reaction region within a particle core, and

FIG. 5 shows a particle together with a drug which can be released in dependence on an extracorporeal signal.

Parts or parameters which correspond to each other are identified with the same reference numbers in all the figures. The features of the particle are depicted symbolically in FIG. 5. Considerations connected with embodiments of the invention are explained with the aid of the remaining figures.

### DETAILED DESCRIPTION OF THE PREFERRED EMBODIMENTS

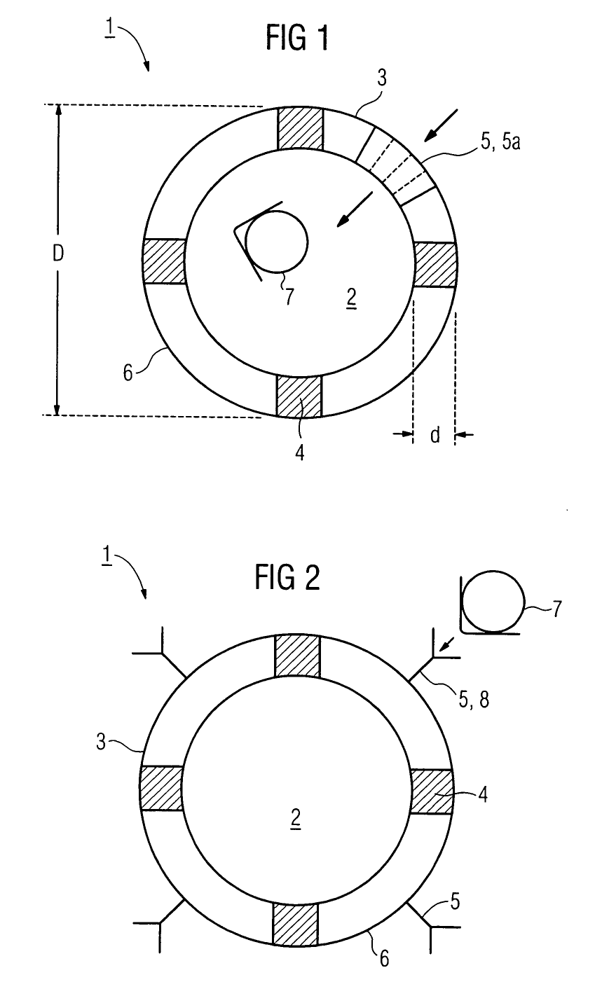

In each case, FIGS. 1 to 5 show, in various embodiments, a particle 1 which can be transported in a human bloodstream. All the functions which are explained with the aid of FIGS. 1 to 5 can also be combined, in an arbitrary manner, in a single particle 1.

The particle 1 is in principle composed of a particle core 2 and of a membrane 3 which surrounds the core. The membrane 3 possesses, as scaffolding, a matrix 4 into which functional elements 5, which are explained in more detail below, can be integrated. In this connection, integration is also understood as meaning the external attachment of a functional element 5 (FIG. 2).

When one or more functional elements 5 are attached externally, the membrane 3 can also be formed completely from the matrix 4. In the exemplary embodiments shown in FIGS. 1 to 5, in each case several membrane elements 6, in some of which functional elements 5 are in turn embedded, are integrated into the matrix 4. However, it is likewise also possible for functional elements 5 to be integrated directly into the matrix 4. Furthermore, the membrane 3 can also be formed exclusively from one or more functional elements 5.

The membrane 3 is preferably completely or predominantly formed from a polymer layer, preferably from polylactides, silicates, rubber or plastic. The membrane 3 can also be formed from biological and/or synthetic tissue.

When it is formed from endogenous human cells, the latter can in each case form individual membrane elements 6. The cells which are used for preparing the membrane 3 are lysed and linked to each other using cementing particles, such as ligases, adhesives or microorganisms, with the formation of the matrix 4. In a comparable manner to the situation in intestinal cells, what are termed tight junctions can be formed, as functional elements 5, between individual cell membrane pieces, each of which forms a membrane element 6.

For applications in human medicine, the particles 1 can be administered in any arbitrary manner, for example orally, nasally, transdermally, or by way of the lungs or the peritoneum. The transport, which is subsequently explained in more detail, of substances through the membrane 3, or an accumulation of substance on the membrane 3, can take place using any forces which can be used in the microscopic range. Those which are in particular to be mentioned in this connection are electroosmotic forces, electrostatic bonding and subsequent polarization (change of direction), biological changes in potential (synapses, ion channels), and also promoter-induced processes or processes which are induced by a transporter gene (cf. reporter gene assays). It is also possible to use mechanical swelling (push/pull) or a port/antiport mechanism. The particle 1 can be influenced by external forces or fields, i.e. signals which are generated outside the human or animal body which is to be treated or observed (FIG. 5).

In that which follows, the exemplary embodiments depicted in the figures will be dealt with individually in more detail. FIGS. 1 and 2 in each case show a particle 1 which is intended for gathering or accumulating an endogenous substance 7. The particle 1, which is also termed a nanoparticle and which can be transported in the bloodstream of a patient, is in both cases intended for diagnostic purposes.

In both cases, the particle core 2 is initially empty or is filled with an aqueous solution, for example. The particle 1, which does not necessarily have a spherical form, has an external diameter D of, for example, 3 μm. The membrane 3 has a thickness d of, for example, 5 nm.

In the exemplary embodiment shown in FIG. 1, a functional element 5, which is to be designated a portal element and whose thickness does not necessarily correspond to the thickness d of the remaining membrane 3, is embedded in the membrane 3. At least a portion of the functional element 5 can also be a component of the particle core 2.

By way of example, FIG. 1 only depicts a single portal element 5; however, several portal elements 5 can also, in a similar manner, be integrated into the membrane 3 or form this membrane. The portal element 5 is selectively permeable, from the exterior toward the interior, for the endogenous substance 7.

Consequently, in the course of using the particle 1, the endogenous substance 7 is collected in the particle core 2. The high concentration of the endogenous substance 7 which is thus obtained within the particle 1 substantially facilitates detection of the endogenous substance 7. In particular, as a result of this accumulation of the endogenous substance 7, it is then possible, if the substance contains iron, for example, to use medicoinstrumental diagnostic methods which are known per se, such as magnetic resonance methods (NMR) to detect the endogenous substance 7 even though the latter is present in the blood of the patient, for example, at a concentration which is so low that it would not otherwise be possible to use an imaging method of this nature. As a rule, the lifetime in the human or animal body of the particles 1 which are loaded with the endogenous substance 7 should be limited. For this purpose, the membrane 3 is composed of substances which can be attacked by enzymes in the body.

The exemplary embodiment shown in FIG. 2 differs from the exemplary embodiment shown in FIG. 1 in that the endogenous substance 7 is attached outside the particle 1. In this exemplary embodiment, the particle core 2 can be dispensed with. Coupling elements 8 are provided for attaching the endogenous substance 7, with these elements at the same time having the function of detectors which respond to the endogenous substance 7. The attachment of the endogenous substance 7 to the particle 1 only increases the diameter D of the latter to an insignificant extent such that there is virtually no effect on the transportability of the particle 1. None of the illustrations is to scale.

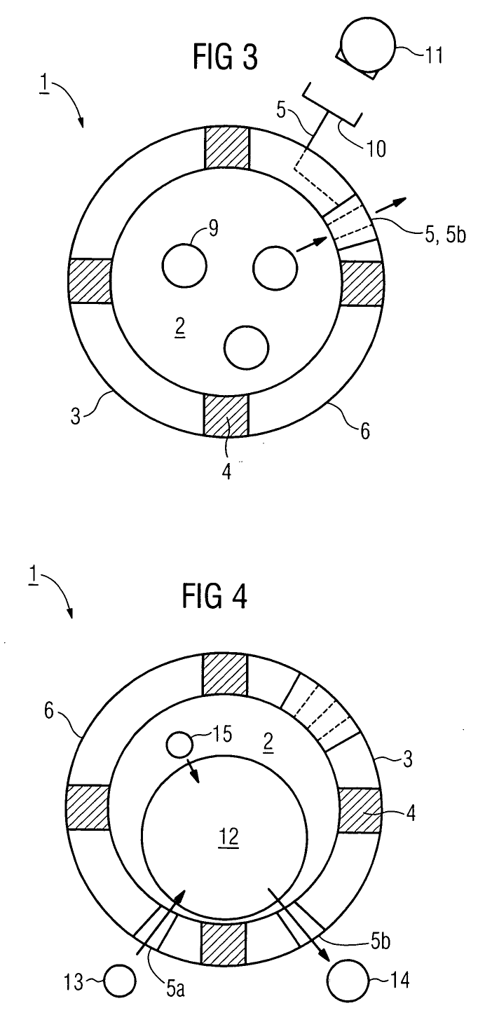

In the exemplary embodiment shown in FIG. 3, a drug 9, which can be secreted out of the particles 1 through a portal element 5, as a functional element, is present in the particle core 2. The portal element 5 cooperates, as indicated by a broken line, with a detector element 10 as an additional functional element 5. However, in contrast to the coupling element 8 depicted in FIG. 2, the detector element 10 is not primarily intended for substance accumulation.

The task of the detector element 10 is simply to detect the presence of a body substance 11 and, as a consequence of this, to establish the permeability of the portal element 5 for the drug 9. In connection with this process, the body substance 11 does not necessarily remain attached to the detector element 10. For example, the detector element 10 can be a potassium receptor which takes up potassium as the body substance 11.

The portal element 5 which is also designated a transport receptor, is coupled to the potassium receptor 10 such that the drug 9 is released. In this way, the drug 9 is supplied selectively to potassium-rich tissue. In particular, if the drug 9 which is present in the particle 1 is only a single particle, the membrane 3 can be constituted such that it is destroyed in conjunction with the release of the drug 9.

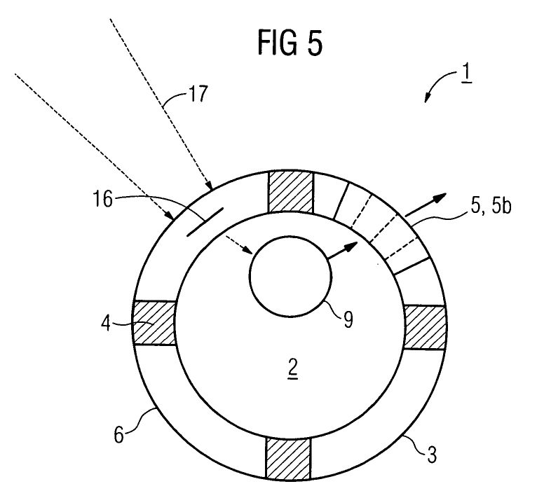

FIG. 4 shows a particle 1 which possesses a reaction region 12 within the particle core 2. As a departure from the illustration, the reaction region 12 can also form the entire particle core 2. This can thereby form what is termed a metabolizing compartment.

In the exemplary embodiment, the membrane 3 possesses two functional elements which, as ingesting element 5a and as secreting element 5b, are provided for internalizing and, respectively, externalizing a substance in and, respectively, out of the particle 1. The functional elements 5 have in each case combined detector and transporter functions. The ingesting element 5a is used for taking up an endogenous intermediate 13 into the reaction region 12.

The endogenous intermediate 13 is a substance which is produced by the body of the patient itself and which is to be further transformed in the body into an end substance 14. It may be imagined that this endogenous transformation is impaired. The corresponding function is assumed by the reaction region 12 using a reaction substance 15.

In the symbolic sketch of the exemplary embodiment, the reaction substance 15 is initially located outside the reaction region 12 and only enters this region when triggered by the presence of the intermediate 13 in the particle core 2. As a departure from this symbolic illustration, the intermediate 13 can, for example, also partially or completely form the reaction region 12 or the particle core 2.

The particle 1 can be designed for the single transformation, or several consecutive transformations, of an endogenous intermediate 13, or of endogenous intermediates 13, respectively, into an end substance 14. In the former case, the membrane 13 can be constituted such that it disintegrates in conjunction with the formation of the end substance 14. The intermediate 13 can also form at least a part of the membrane 3. Taken overall, the particle 1 constitutes a nanofactory, for example like an artificial ribosome, which exclusively produces and releases given reaction products, in particular active compounds.

In particular, the particle 1, together with the reaction region 12 which is enclosed by the membrane 3, as symbolized in the exemplary embodiment shown in FIG. 4, is suitable for the tissue-selective administration of a genetic medicament. In this connection, the genetic medicament, as drug 9, can be produced in the target tissue, only with production of the medicament being induced by the given reaction conditions and/or by way of selective external influences. There is likewise also the possibility, however, of releasing a diagnostic agent exclusively or predominantly in a target tissue.

FIG. 5 shows an exemplary embodiment in which a particle 1 can be connected physically and/or by data provision to external systems. As in the case of the exemplary embodiment shown in FIG. 3, the particle core 2 contains a drug 9 which can be secreted from the membrane 3 through a functional element 5. In addition to this, the membrane 3 possesses a recipient element 16 which can be actuated by an external signal 17, for example an ultrasonic signal or an electromagnetic signal.

The actuation of the membrane 3, which can be influenced from the exterior, takes place using what is termed membrane exchange software (MES). When actuation takes place using an electromagnetic signal, the breadth of the recipient element 16 should be approximately of the order of size of, or somewhat less than, the wavelength of the external signal 17.

If, for example, light, in particular infrared light, is used as the external signal 17, this requirement can then be met using a recipient element 16 which is in or below the micrometer range. As indicated by arrows in the illustration, the recipient element 16 is functionally coupled to the portal element 5. As a departure from the illustration, it is also possible to combine the detector or recipient element 16 with the portal element in a single functional element 5.

In this case, the action of the external signal 17 can, for example, open the portal element 5 such that the latter renders possible particle transport from the interior to the exterior, as in the exemplary embodiment shown in FIG. 5, or particle transport from the exterior to the interior, as in the exemplary embodiment shown in FIG. 1. The possibility of combining an externally induced particle transport through the membrane 3 with imaging medicoinstrumental methods is particularly advantageous.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A particle, transportable in a fluid stream, comprising:
a membrane enclosing a particle core, the membrane including a plurality of functional elements, integrated in a matrix, which, in dependence on the concentration of a body substance, bring about at least one of substance transport through, and substance accumulation at, the membrane, wherein a detector element is provided as a functional element, and wherein the at least one of substance accumulation at, and the substance passage through, the membrane is influenceable by an extracorporeal signal provided for actuating the detector element.

2. The particle as claimed in claim 1, wherein the particle is transportable in at least one of a human and animal bloodstream.

3. The particle as claimed in claim 1, wherein the membrane accumulates an endogenous substance.

4. The particle as claimed in claim 1, wherein release of a drug through the membrane depends on the concentration of the body substance.

5. The particle as claimed in claim 1, wherein the membrane is attackable by enzymes in the body.

6. The particle as claimed in claim 1, wherein the particle core includes a reaction region, intended for substance transformation.

7. The particle as claimed in claim 6, wherein the reaction region is intended for transforming an endogenous intermediate.

8. The particle as claimed in claim 1, wherein the external diameter is at least 50 nm and at most 10 μm.

9. The particle as claimed in claim 1, wherein the thickness of the membrane is at least 2 nm and at most 1 μm.

10. The particle as claimed in claim 1, wherein the extracorporeal signal is an ultrasonic signal.

11. The particle as claimed in claim 10, wherein the extracorporeal signal is an electromagnetic signal.

12. The particle as claimed in claim 1, wherein a portal element, which enables substance transport to take place through the matrix, is provided as a functional element.

13. The particle as claimed in claim 12, wherein the detector element is functionally coupled to the portal element.

14. The particle as claimed in claim 1, wherein the matrix is formed from a polymer layer.

15. A method for detecting a particle as claimed in claim 1, comprising:
detecting the change in the particle, brought about by at least one of the substance transport through, and the substance accumulation at, the membrane, in an imaging medicoinstrumental method.

16. The particle as claimed in claim 2, wherein the membrane accumulates an endogenous substance.

17. The particle as claimed in claim 2, wherein release of a drug through the membrane depends on the concentration of the body substance.

18. The particle as claimed in claim 2, wherein the membrane is attackable by enzymes in the body.

19. The particle as claimed in claim 2, wherein the particle core includes a reaction region, intended for substance transformation.

20. The particle as claimed in claim 3, wherein release of a drug through the membrane depends on the concentration of the body substance.

21. The particle as claimed in claim 3, wherein the membrane is attackable by enzymes in the body.

22. The particle as claimed in claim 3, wherein the particle core includes a reaction region, intended for substance transformation.

23. The particle as claimed in claim 19, wherein the reaction region is intended for transforming an endogenous intermediate.

24. The particle as claimed in claim 2, wherein the external diameter is at least 50 nm and at most 10 μm.

25. The particle as claimed in claim 2, wherein the thickness of the membrane is at least 2 nm and at most 1 μm.

26. The particle as claimed in claim 2, wherein the extracorporeal signal is an ultrasonic signal.

27. The particle as claimed in claim 2, wherein the matrix is formed from a polymer layer.

28. The particle as claimed in claim 1, wherein the fluid stream is a bloodstream.
